(19) United States
(12) Patent Application Publication
(10) Pub. No.: US 2015/0250700 A1
(43) Pub. Date: Sep. 10, 2015

(54) COMPOSITION FOR EXTERNAL USE SKIN PREPARATION, CONTAINING THIOREDOXIN

(71) Applicant: AMOREPACIFIC CORPORATION, Seoul (KR)

(72) Inventors: Ji Yeong Kim, Yongin-si (KR); Joon Young Hwang, Yongin-si (KR); Young So Kim, Yongin-si (KR); Sang Hoon Han, Yongin-si (KR)

(73) Assignee: AMOREPACIFIC CORPORATION, Seoul (KR)

(21) Appl. No.: 14/431,853

(22) PCT Filed: Nov. 13, 2013
(86) PCT No.: PCT/KR2013/010256

§ 371 Date: Mar. 27, 2015

(30) Foreign Application Priority Data
Nov. 13, 2012 (KR) 10-2012-0127891

(51) Int. Cl. A61K 8/66; A61Q 19/08; A61Q 19/00

(52) U.S. Cl. CPC A61K 8/66; A61Q 19/007; A61Q 19/08; A61Q 19/00

(57) ABSTRACT

The present invention relates to a composition for an external use skin preparation, containing thioredoxin, and more specifically, to a composition which contains thioredoxin thereby providing an overall improvement in skin condition such as a remarkable improvement in skin moisturization, sebum control, pore contraction, an improvement in skin color through blood circulation improvement, and the like.

### TECHNICAL FIELD

The present invention relates to a composition for skin external application containing thioredoxin, and more particularly, to a composition containing thioredoxin that can provide an excellent effect of improving overall skin conditions by improving skin moisturization, controlling sebum, tightening pores, and improving blood circulation to improve complexion.

### BACKGROUND ART

The skin is the primary barrier of the human body, which functions to protect the organs of the body from external environmental stimuli such as changes in temperature and humidity, UV rays and pollutants, and undergoes changes with aging due to a variety of intrinsic and extrinsic factors. Specifically, with respect to the intrinsic factors, the secretion of various hormones that regulate metabolisms decreases, and the function of immune cells and the activity of cells decrease, so that the biosynthesis of immune proteins required for the body and constituent proteins of the body decreases. With respect to the extrinsic factors, as the amount of ultraviolet rays reaching the earth's surface is increasing due to destruction of the ozone layer, and as environmental pollution becomes ever more serious, free radicals and reactive oxygen species increase. As a result, various changes in the skin occur, including reduced thickness, increased wrinkles, reduced skin elasticity, dark skin color, frequent occurrence of skin troubles, increased age spots, freckles and dark spots, bad complexion becomes bad, and darker skin tone.

To prevent changes in skin conditions from being caused by such intrinsic and extrinsic factors and to keep the skin more healthy and elastic, there have been efforts to use cosmetics containing physiologically active substances obtained from various animals, plants and microorganisms, thereby improving skin conditions.

### PRIOR ART DOCUMENTS

Patent Document 1: Korean Patent Registration No. 0585269

### DISCLOSURE

### Technical Problem

Accordingly, the present inventors have found that thioredoxin, an enzyme essential for life activity, is highly safe for the skin, because it is a protein that is actually expressed in the skin, and thioredoxin can provide an excellent effect of improving skin conditions when it is applied to the skin, thereby completing the present invention.

Therefore, it is an object of the present invention to provide a composition for skin external application, which contains thioredoxin capable of improving overall skin conditions.

### Technical Solution

In order to accomplish the above objects, the present invention provides a skin external composition for skin moisturization, which contains thioredoxin as an active ingredient.

The present invention also provides a skin external composition for improving complexion and skin tone, which contains thioredoxin as an active ingredient.

The present invention also provides a skin external composition for pore tightening, which contains thioredoxin as an active ingredient.

The present invention also provides a skin external composition for controlling sebum, which contains thioredoxin as an active ingredient.

### Advantageous Effects

The composition of the present invention contains thioredoxin that can provide the effect of improving overall skin conditions by improving skin moisturization, controlling sebum, tightening pores, and improving blood circulation to improve complexion.

### DESCRIPTION OF DRAWINGS

FIG. 1 shows a process of obtaining thioredoxin from Saccharomyces ferment.

### BEST MODE

A composition for skin external application according to the present invention contains thioredoxin as an active ingredient.

Thioredoxin is a low-molecular-weight protein having a molecular weight of 10,000-13,000, and is also abbreviated as TRX. Thioredoxin acts as a proton donor when ribonucleotide reductase reduces ribonucleotides. A pair of cysteine residues present in the active center of thioredoxin is conserved in prokaryotes and eukaryotes, and thioredoxin has the capability to reduce and cleave the disulfide bond of a target protein in the presence of NADPH and thioredoxin reductase. Also, it is known that human TRX/ADF (human thioredoxin/adult T cell leukemia-derived factor) is involved in cell proliferation or the control of transcription factors.

Thioredoxin that is used in the present invention may be isolated by a method known in the art. Preferably, a substance comprising thioredoxin may be cultured by fermentation. Particularly, thioredoxin that is used in the present invention may be isolated from a filtrate obtained by filtration of ferment of yeast, preferably yeast of the genus Saccharomyces.

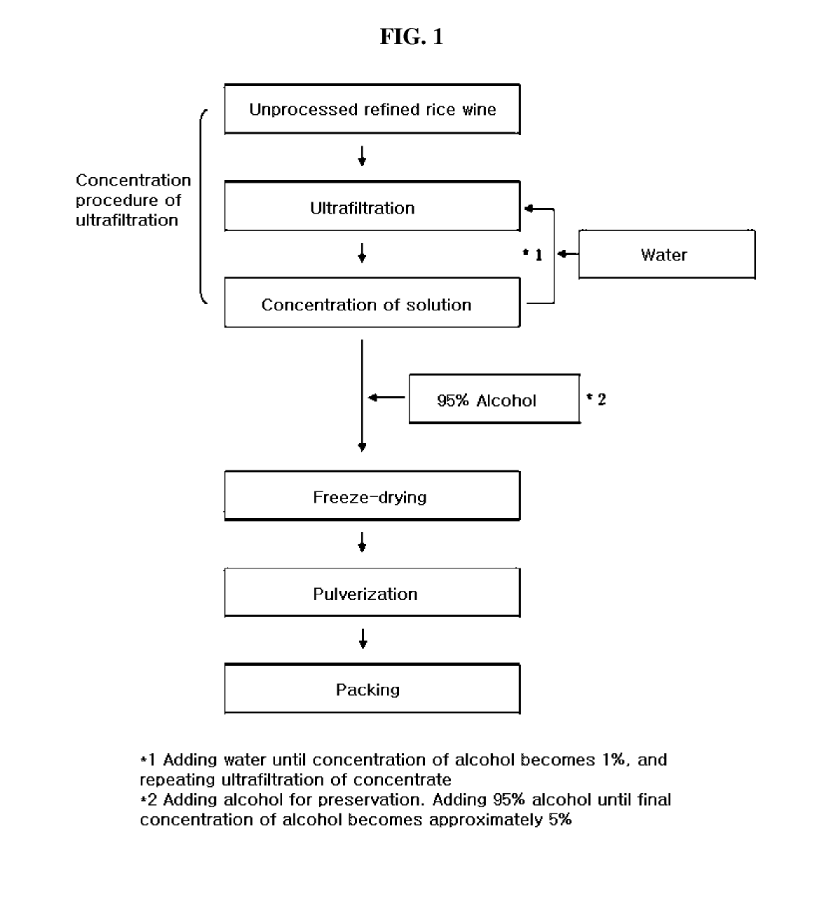

FIG. 1 shows a process of obtaining thioredoxin, which is used in the present invention, from Saccharomyces fermentation broth.

The composition according to the present invention may contain thioredoxin in an amount of 0.00001-50 wt %, preferably 0.00001-30 wt %, and more preferably 0.00001-10 wt %, based on the total weight of the composition. If the content of thioredoxin in the composition is less than 0.00001 wt %, the efficacy and effects of thioredoxin will be insignificant, and if the content of thioredoxin is more than 50 wt %, it will cause problems in terms of skin safety and formulation.

The composition of the present invention may be used as a skin external composition for skin moisturization, which can enhance the skin barrier function and induce the differentiation of skin keratinocytes. Thus, it can be effectively used as a skin external composition for preventing or ameliorating dry skin, contact dermatitis or psoriasis, which result from imperfect epidermal differentiation.

The composition of the present invention may be used as a skin external composition for improving complexion and skin tone. When it is applied to the skin, it will exhibit excellent effects of enlarging capillary blood vessels and promoting blood circulation to facilitate skin nourishment, and inhibiting skin aging to improve complexion and skin tone.

The composition of the present invention may be used as a skin external composition for tightening pores, controlling sebum and reducing skin trouble. When it is applied to the skin, it will exhibit excellent effects of inhibiting the excessive secretion of sebum, promoting reactive oxygen species elimination and collagen synthesis to tighten pores, and reducing the expression of inflammatory factors to inhibit skin trouble. In addition, it can inhibit skin irritation due to its high antioxidant activity.

The skin external composition according to the present invention may be formulated as a cosmetic composition, and may contain a cosmetically and dermatologically acceptable medium or base. The composition may be formulated as a preparation for topical application. Examples of formulations for topical application include a solution, a gel, a solid, a paste anhydride, an emulsion prepared by dispersing an oil phase in a water phase, a suspension, a microemulsion, microcapsules, microgranules, ionic (liposome) and non-ionic vesicles, cream, toner, lotion, powder, an ointment, a spray, and a conceal stick. Also, the composition according to the present invention may be formulated as a foam composition or an aerosol composition further containing a compressed propellant. In addition, the composition of the present invention may be formulated according to a conventional method known in the art.

Further, the composition according to the present invention may contain additives which are generally used in the cosmetic field or the dermatological field, for example, fatty substance, organic solvent, solubilizing agent, thickener, gelling agent, softener, antioxidant, suspending agent, stabilizer, foaming agent, aromatic, surfactant, water, ionic or non-ionic emulsifying agent, filler, sequestering agent, chelating agent, preservative, vitamins, blocking agent, wetting agent, essential oil, dye, pigment, hydrophilic or hydrophobic activator, lipid vesicle, or other components which are generally used in cosmetics. These additives are contained in amounts which are generally used in the cosmetic field or the dermatological field.

In addition, the composition of the present invention may contain a skin absorption-promoting substance in order to increase the effect of improving skin conditions.

The composition of the present invention is not specifically limited, and can be suitably selected according to the intended use. For example, it may be formulated as skin care products such as toner, lotion, essence, cream, ointment, gel, pack, patch, mask and spray products, makeup products such as makeup base, foundation, powder, mascara and lipstick products, cleanser products such as cleaning oil, cleaning cream, cleansing gel and point makeup remover products, etc.

### MODE FOR INVENTION

Hereinafter, the constitution and effects of the present invention will be described in further with reference to test examples and formulation examples. It is to be understood, however, that these test examples and formulation examples are for illustrative purposes only and are not intended to limit the scope of the present invention.

### Reference Example 1

Thioredoxin used to test the effects of the composition according to the present invention is a TRX rice wine extract (Saccharomyces ferment) manufactured by Pharma Food International Company Ltd. (1-49, Goryo-Ohara, Nishikyo-ku, Kyoto, 615-8245 Japan), and has a thioredoxin content of 4 mg/g.

### Formulation Example 1 and Comparative Formulation Example 1

According to the compositions shown in Table 1 below, nourishing creams were prepared (unit: wt %).

TABLE 1

| | Formulation | Comparative Formulation |
|---|---|---|
| Components | Example 1 | Example 1 |
| Purified water | To 100 | To 100 |
| Thioredoxin | 0.1 | — |
| Vegetable hydrogenated | 1.50 | 1.50 |
| oil | | |
| Stearic acid | 0.60 | 0.60 |
| Glycerol stearate | 1.00 | 1.00 |
| Stearyl alcohol | 2.00 | 2.00 |
| Polyglyceryl-10 | 1.00 | 1.00 |
| pentastearate & behenyl | | |
| alcohol & sodium | | |
| stearoyl lactylate | | |
| Arachidyl behenyl | 1.00 | 1.00 |
| alcohol & arachidyl | | |
| glucoside | | |
| Cetyl aryl alcohol & | 2.00 | 2.00 |
| cetearyl glucoside | | |
| PEG-100 stearate & | 1.50 | 1.50 |
| glycerol oleate & | | |
| propylene glycol | | |
| Caprylic/capric | 11.00 | 11.00 |
| triglyceride | | |
| Cyclomethicone | 6.00 | 6.00 |
| Preservative and | q.s. | q.s. |
| fragrance | | |
| Triethanolamine | 0.1 | 0.1 |

### Test Example 1

### Measurement of the Effect of Increasing Skin Moisturization

In order to measure the effect of thioredoxin on an increase in skin moisturization, the creams of Formulation Example 1 and Comparative Formulation Example 1 were used and evaluated in the following manner.

Twenty adult men and women, who were 40-50 years old and had dry skin, were divided into the following two groups: a group to which the nourishing cream of Formulation Example 1 was applied; and a group to which the nourishing cream of Comparative Formulation Example 1 was applied. Each nourishing cream was applied to the face twice a day for 4 weeks. Before the start of the application, at 1, 2 and 4 weeks after the start of the application and at 2 weeks after the stop of the application (6 weeks after the start of the application), the skin moisture content was measured using a Corneometer (CM825, C+K Electronic GmbH, Germany) under constant temperature and constant humidity conditions (temperature: 24° C., and relative humidity: 40%). The results of the measurement are shown in Table 2 below. The results in Table 2 are expressed as the percent increase in the moisture content after application relative to the moisture content measured immediately before the start of application.

TABLE 2

Increase (%) in moisture content

| | After | After | After | After |
|---|---|---|---|---|
| Test groups | 1 week | 2 weeks | 4 weeks | 6 weeks |
| Formulation | 31 | 33 | 36 | 33 |
| Example 1 | | | | |
| Comparative | 30 | 32 | 32 | 15 |
| Formulation | | | | |
| Example 2 | | | | |

As can be seen from the results in Table 2, in the case in which the cream of Comparative Formulation Example 1 was applied, an increase in skin moisture content of about 30% appeared up to 4 weeks after the application, but the skin moisture content decreased after the stop of the application. However, in the case in which the cream of Formulation Example containing thioredoxin was applied, an increase in skin moisture content of 30% or more appeared even after the stop of the application. This suggests that the thioredoxin-containing composition of the present invention has an excellent skin moisturizing effect.

### Test Example 2

### Measurement of the Effect of Stimulating the Differentiation of Keratinocytes

In order to examine the effect of thioredoxin on the stimulation of differentiation of keratinocytes, the amount of cornified envelope (CE) protein produced during the differentiation of keratinocytes was measured by absorbance in the following manner.

Specifically, human keratinocytes, isolated from the dermis of newborns and primarily cultured, were placed in a culture flask and attached to the bottom. Then, the cells were cultured with a medium containing 5 ppm of thioredoxin for 5 days until the cells reached to a confluence of about 70-80%. At the same time, a low calcium (0.03 mM)-treated group and a high calcium (1.2 mM)-treated group were used as a negative control group and a positive control group, respectively. Then, the cultured cells were harvested and washed with PBS (phosphate buffered saline). Then, the cells were sonicated in 1 ml of 10 mM Tris-HCl (pH 7.4) containing 2% SDS (sodium dodecyl sulfate) and 20 mM DTT (dithiothreitol), boiled and centrifuged. The precipitate was suspended in 1 ml of PBS, and the absorbance at 340 nm was measured. Separately, a portion of the solution following the sonication was taken, and the protein content thereof was measured as a reference for evaluating the degree of differentiation of the cells. The results of the measurement are shown in Table 3 below.

TABLE 3

| | Degree (%) of differentiation |
|---|---|
| Test samples | of keratinocytes |
| Low-calcium (0.03 mM) solution | 100 |
| (negative control) | |
| High-calcium (1.2 mM) solution | 210 |
| (positive control) | |
| Thioredoxin | 295 |

As can be seen in Table 3 above, treatment with thioredoxin shows an excellent effect of stimulating the differentiation of keratinocytes.

### Test Example 4

### Measurement of the Effect of Restoring Skin Barrier Function

In order to measure the effect of thioredoxin on the restoration of the skin barrier function impaired due to skin damage, the following experiment was performed.

The skin barrier of the upper arm of each of 10 adult men and women was impaired using a tape stripping method, and each of the compositions of Formulation Example 2 and Comparative Formulation Example 2, shown in Table 4 below, was applied to the impaired portion while the degree of restoration of transepidermal water loss (TEWL) was measured using Vapometer (Delfin, Finland) once a day for 7 days. Herein, Comparative Formulation Example 2 is a vehicle as a negative control. The results of the measurement are shown in Table 5 below. The results in Table 5 are expressed by a percentage based on a difference between before and after impairment being 100%.

TABLE 4

| | Formulation | Comparative Formulation |
|---|---|---|
| Components | Example 2 | Example 2 |
| Purified water | 69 | 70 |
| Propylene glycol | 30 | 30 |
| Thioredoxin | 1 | — |

TABLE 5

| | Change (%) in TEWL | | | | | | |
|---|---|---|---|---|---|---|---|
| | Before | | | | 4 | 5 | 6 |
| Test groups | treatment | 1 day | 2 days | 3 days | days | days | days |
| Formulation | 100 | 126.8 | 128.5 | 122.8 | 116.8 | 111.2 | 108.5 |
| Example 2 | | | | | | | |
| Comparative | 100 | 121.4 | 112.7 | 98.3 | 70.5 | 62.3 | 43.5 |
| Formulation | | | | | | | |
| Example 2 | | | | | | | |

As can be seen in Table 5 above, when the skin was treated with Comparative Formulation Example 2 containing no thioredoxin, transepidermal water loss gradually increased with the passage of time. However, when the skin was treated with Formulation Example 2 containing thioredoxin, transepidermal water loss was quickly restored to normal level, and barrier impairment was restored.

### Test Example 5

### Effect on Improvement in Complexion

In order to evaluate the effect of the cosmetic composition according to the present invention on the promotion of skin blood circulation, the degree of blood circulation in the skin was measured using a laser doppler perfusion imager (LDPI). The LDPI is widely known as a device for measuring blood circulation in the skin and is a very sensitive device capable of measuring not only the velocity and amount of blood in the capillary vessel of the skin, but also blood flow in arterioles and venules.

In a constant-temperature and constant-humidity chamber, the face was washed with soap and adapted for 30 minutes, and initial values were measured using LDPI. 30 women whose hands and feet were usually cold participated in the test, and the initial blood flow rate in the portion below the forehead of the participants was measured using LDPI. Next, the compositions of Formulation Example 1 and Comparative Formulation Example 1 were applied to the subjects for one week, and then the blood flow rates were compared with the initial measurement values, and the results of the comparison (change in skin blood rate) are shown in Table 6 below.

TABLE 6
LDPI results before and after use of cosmetic
compositions- skin blood rate

| | Change (%) in skin blood rate after |
|---|---|
| Test samples | one week application |
| Formulation Example 1 | 12 |
| Comparative Formulation Example 1 | 5 |

As can be seen from the results in Table 6 above, the cosmetic composition of the present invention significantly increased the skin blood rate compared to the formulation of Comparative Example 1 containing no thioredoxin, suggesting that the composition of the present invention improves complexion by stimulating blood circulation. This ultimately suggests that the thioredoxin-containing composition of the present invention can contribute to the effective transfer of nutrients to the skin and the inhibition and delay of aging.

### Test Example 6

### Effect on Improvement in Skin Tome

In order to examine the effects of the formulations of Formulation Example 1 and Comparative Example 1 on improvement in skin tone, each of the formulations of Formulation Example 1 and Comparative Formulation Example 1 was applied to 30 subjects in the evening once a day for one week, and then the degree of skin tone improvement was evaluated using Facial Stage DM-3 (Moritex, Japan). The degree of skin tone improvement was determined based on the changes in the brightness and saturation values of the skin. The results are shown in Table 7 below. In Table 7, greater changes in the brightness and saturation values indicate greater improvement in skin tone.

TABLE 7

| | Skin tone improvement (%) | |
|---|---|---|
| | Brightness | Saturation |
| Test samples | (mean ± SD) | (mean ± SD) |
| Formulation Example 1 | 15 ± 3.24 | 12 ± 2.34 |
| Comparative Formulation Example 1 | 5 ± 2.34 | 5 ± 2.05 |

As can be seen from the results in Table 7 above, the formulation of Comparative Formulation Example 1 containing no thioredoxin showed no significant effect on skin tone improvement, whereas the formulation of Formulation Example 1 containing thioredoxin as an active ingredient showed a significant improvement in skin tone after application compared to before application.

### Test Example 7

### Pore Tightening Effect

1. Effect of Tightening Pores by Stimulation of Collagen Biosynthesis

The effect of thioredoxin of the present invention on the stimulation of collagen biosynthesis was measured in comparison with TGF-beta.

First, fibroblasts were seeded into a 24-well plate at a density of 10⁵ cells/well and cultured in serum-free DMEM medium for 24 hours to a confluence of about 90%. Then, the cells were treated with each of a solution of thioredoxin of the present invention and 10 ng/ml of TGF-beta dissolved in serum-free medium and incubated in a CO₂ incubator for 24 hours. The supernatants of the cell cultures were collected and the amount of procollagen therein was measured using a procollagen type (I) ELISA kit. The results of the measurement are shown in Table 8 below. The values of collagen synthesis (%) in Table 8 are expressed as percentages relative to the control taken as 100%.

TABLE 8

| Test samples | Collagen synthesis (%) |
|---|---|
| Control | 100 |
| TGF-β | 183.5 |
| Thioredoxin | 196.2 |

As can be seen from the results in Table 8 above, thioredoxin according to the present invention showed a high ability to synthesize collagen compared to the positive control TGF-beta. This suggests that thioredoxin according to the present invention can tighten pores by increasing the production of collagen around pores.

### 2. Pore Tightening Effect

The pore tightening effects of the nourishing cream formulations of Formulation Example 1 and Comparative Formulation Example 1 were evaluated in the following manner. Specifically, 20 men and women having large pore size were selected and divided into two groups, each consisting of 10 people. Each of the nourishing cream formulations of Formulation Example 1 and Comparative Formulation Example 1 was applied to the face every day for 4 weeks. To determine the effect of tightening pores, photographs were taken before application and after 4 weeks of application and visually evaluated by experts. The evaluation was made on a six-point scale (0 to 5; 0: not tightened; 5: very tightened), and the results of the evaluation are shown in Table 9 below.

TABLE 9

| Test samples | Score |
|---|---|
| Formulation Example 1 | 4 |
| Comparative Formulation Example 1 | 0 |

As can be from the results in Table 9 above, the nourishing cream formulation of Comparative Formulation Example 1 had no effect on the tightening of pores, but the cream formulation of Formulation Example 1 showed a visible effect on the tightening of pores, suggesting that thioredoxin according to the present invention has an excellent effect of reducing pore size.

### Test Example 8

### Serum Secretion Inhibitory Effect

1. Effect of Inhibiting Skin Excessive Secretion of Sebum by Inhibition of 5α-Reductase Activity

In order to examine the effect of inhibiting 5α-reductase activity, the ratio of [¹⁴C]testosterone-to-[¹⁴C]dihydrotestosteron conversion in HEK293-5αR2 cells was measured. HEK293 cells transfected with p3×FLAG-CMV-5αR2 were added to a 24-well plate at a density of 2.5×10⁵ cells/well and cultured (Park et al., 2003, JDS. Vol. 31, pp. 191-98). The next day, the medium was replaced with a fresh medium containing an enzyme substrate and an inhibitor. As the substrate of the medium, 0.05 μCi [¹⁴C]testosterone (Amersham Pharmacia Biotech, UK) was used.

In order to measure the degree of inhibition of 5α-reductase activity, thioredoxin was added to the cells which were then incubated in a 5% CO₂ incubator at 37° C. for 2 hours. At the same time, a medium containing no thioredoxin was used as a negative, and a medium containing finasteride was used as a positive control. Next, the culture medium was collected, and steroid was extracted with 800 μl of ethyl acetate. The upper organic solvent layer was separated and dried, and the remaining material was dissolved in 50 μl of ethyl acetate and developed on silica plastic sheet kieselgel 60 F254 using ethyl acetate-hexane (1:1) as a developing solvent.

The plastic sample was dried in air, and the amount of isotopes was measured using a Vas system. Specifically, the dried plastic sheet together with an X-ray film was placed in a Vas cassette, and after 1 week, the amounts of the testosterone and dihydrotestosterone isotopes were measured, and then the percent conversion and the percent inhibition were measured using the following equations 1 and 2, respectively. The results of the measurement are shown in Table 10 below.

Conversion (%)=radioactivity in the DHT region/total radioactivity×100 Equation 1

Inhibition (%)=(conversion of control−conversion of sample)/conversion of control×100 Equation 2

TABLE 10

| Test samples | Conversion (%) | Inhibition (%) |
|---|---|---|
| Negative control | 48.0 | — |
| Positive control | 27.6 | 42.5 |
| Thioredoxin | 15.9 | 60.8 |

As can be seen from the results in Table 10 above, thioredoxin of the present invention blocks the conversion of testosterone to dihydrotestosterone by effectively inhibiting the activity of 5α-reductase that converts testosterone to dihydrotestosterone to enter the nucleus by binding to receptor protein in the cytoplasm so as to activate sebaceous gland cells and stimulate the differentiation of the cells to induce excessive secretion of sebum from sebaceous glands. Also, it was shown that thioredoxin had an excellent inhibitory effect compared to finasteride known to inhibit 5α-reductase activity. This suggests that thioredoxin of the present invention can inhibit excessive secretion of sebum by effectively inhibiting 5α-reductase activity.

2. Sebum Secretion Inhibitory Effect

The effects of the nourishing cream formulations of Formulation Example 1 and Comparative Formulation Example 1 on the inhibition of sebum secretion were evaluated in the following manner. Specifically, 30 men and women in which a large amount of sebum was secreted were selected, and the nourishing cream formulations of Formulation Example 1 and Comparative Formulation Example 1 were applied to the appointed areas every day for 4 weeks. To determine the effect on sebum reduction, average reductions (%) in sebum after 2 weeks and 4 weeks were measured using a sebumeter (SM810, C+K Electronic GmbH, Germany), and the results of the measurement are shown in Table 11 below.

TABLE 11

| | Reduction (%) in sebum | |
|---|---|---|
| Test samples | After 2 weeks | After 4 weeks |
| Formulation Example 1 | 33 | 42 |
| Comparative Formulation Example 1 | 5 | 5 |

As can be seen from the results in Table 11 above, the cream formulation of Formulation Example 1 containing thioredoxin of the present invention as an active ingredient effectively inhibited excessive secretion of sebum compared to the cream formulation of Comparative Formulation Example 1 containing no thioredoxin.

1. A skin external composition, which contains thioredoxin as an active ingredient and a dermatologically acceptable carrier.

2. The skin external composition according to claim 1, which further comprises at least one selected from the group consisting of fatty substance, organic solvent, solubilizing agent, thickener, gelling agent, softener, antioxidant, suspending agent, stabilizer, foaming agent, aromatic, surfactant, water, ionic or non-ionic emulsifying agent, filler, sequestering agent, chelating agent, preservative, vitamins, blocking agent, wetting agent, essential oil, dye, pigment, hydrophilic or hydrophobic activator, and lipid vesicle.

3. The skin external composition according to claim 1, which further comprises skin absorption-promoting substance.

4.-6. (canceled)

7. The skin external composition of claim 1, wherein the thioredoxin is contained in an amount of 0.00001-50 wt % based on the total weight of the composition.

8. The skin external composition of claim 1, wherein the thioredoxin is isolated from a filtrate obtained by filtering yeast ferment.

9. The skin external composition of claim 8, wherein the yeast is of the genus Saccharomyces.

10. A method for moisturizing skin of a subject, comprising applying the composition of claim 1 to skin of the subject.

11. A method for enhancing skin barrier function of skin of a subject, comprising applying the composition of claim 1 to skin of the subject.

12. A method for inducing differentiation of skin keratinocytes in skin of a subject, comprising applying the composition of claim 1 to skin of the subject.

13. A method for improving complexion and skin tone of skin of a subject, comprising applying the composition of claim 1 to skin of the subject.

14. A method for tightening pores of skin of a subject, comprising applying the composition of claim 1 to skin of the subject.

15. A method for controlling sebum of skin of a subject, comprising applying the composition of claim 1 to skin of the subject.
